(19) United States
(12) Patent Application Publication
(10) Pub. No.: US 2009/0311751 A1
(43) Pub. Date: Dec. 17, 2009

(54) Wood-rotting basidiomycetes for production of ligninolytic enzymes

(76) Inventors: Vladimir Elisashvili, Tbilisi (GE); Mordechai Rebhun, Doar-Na Galil Tachton (IL)

Correspondence Address: MARTIN D. MOYNIHAN d/b/a PRTSI, INC., P.O. BOX 16446, ARLINGTON, VA, 22215, US

(21) Appl. No.: 11/919,175

(22) PCT Filed: Apr. 25, 2006
(86) PCT No.: PCT/IL06/00502

§ 371 Date: Oct. 24, 2007

(51) Int. Cl. C12P 21/00; C12N 1/14; C12N 9/04; C12N 9/08; D06M 16/00; D21C 3/00

(52) U.S. Cl. 435/71.2; 435/254.1; 435/190; 435/192; 435/262; 435/263; 435/278

(57) ABSTRACT

The invention relates to the production of ligninolytic enzymes, laccase and manganese peroxidase, from certain white-rot basidiomycetes fungi, using highly efficient fermentation techniques. The aim of this invention is to create a novel economically and time-effective overall procedure comprising use of specific mushroom strains, fermentation process and the isolation-purification techniques, for producing the aforesaid enzymes. In particular, a submerged fermentation of the specific strains on a variety of lignocellulosic substrates from organic wastes like waste of ethanol production from wheat grain, mandarin peels and bran is developed. Culturing conditions can be selected to modify the laccase/manganese peroxidase ratio in favour of the production of either laccase or manganese peroxidase.

### FIELD OF THE INVENTION

The present invention relates to the producing of ligninolytic enzymes like laccase, manganese peroxidase and lignin peroxidase from white-rot fungi.

### BACKGROUND OF THE INVENTION

White-rot fungi are characterized by unique ability to degrade recalcitrant wood polymer-lignin. The major enzymes associated with lignin-degrading ability of white rot fungi are lignin peroxidase (LiP), manganese peroxidase (MnP) and laccase (Lac) although the above fungi do not have the same set of enzymes. Recently, extensive research on these fungi has been conducted with aim to isolate new organisms with increased secretion of ligninolytic enzymes as well as enzymes with properties that are important for industrial applications: in bioremediation of industrial waste streams polluted by hazardous xenobiotics; biobleaching and biopulping; in the textile and dye industries; biotransformation of pharmaceutical and other intermediates; food industry; biosensors construction; cosmetics; medicine and analytic biochemistry. All these biotechnological applications require large amounts of enzyme at low cost. However, the enzymes that are presently manufactured are still expensive due to the low yield and high cost in production and isolation. In addition, although many recombinant organisms efficiently overproduce various industrial enzymes, reliable expressions of laccase and peroxidases in heterologous systems have not been achieved yet and they still have to be obtained from natural sources. Therefore, the major task is to extend the spectrum of ligninolytic enzymes producing organisms and to increase their production by optimising culture conditions, especially by adding specific enzyme inducers.

During the last years, it has frequently been attempted to use white rot fungi for the production of ligninolytic enzymes. Phanerochaete chrysosporium, Trametes and Pleurotus species, Phlebia radiata were the most extensively studied basidiomycetes. Now it is known that the ligninolytic machinery in white rot fungi is highly regulated by nutrients. In particular, carbon and nitrogen sources, as well as several aromatic compounds and microelements, have been shown to have strong regulating effects. On the basis of data 5 received, several approaches were used to promote enzyme production: fungi cultivation under nitrogen or carbon limiting conditions, addition to the nutrient medium of inducers like xylidine, ferulic acid, and veratryl alcohol, addition of ethanol, manganese salts, polypropylene glycol, phospholipids, and unsaturated fatty acids. For example, JAGER et al. (APPLIED AND ENVIRONMENTAL MICROBIOLOGY, 1985, pp. 1274-1278) have proposed adding a detergent, such as TWEEN 80 or TWEEN 20, to the submerged cultures in order to increase the production of lignin peroxidase in proportions comparable to that currently obtained in stationary cultures. LEISOLA et al. (J. BIOTECHNOL., 1985, pp. 97-107) have reported that the addition of veratryl alcohol increases the synthesis of lignin peroxidase. The proposal has been made (U.S. Pat. No. 5,153,121) by ASTHER et al. of a method for producing lignin peroxidase from Phanerochaete chrysosporium comprising the addition of phospholipids, emulsified fatty acids and veratryl alcohol at different steps of culture and by varying the content of constituents. Lignin peroxidase production of 46.4 nkat/ml was obtained in 100 ml of medium being introduced into a 250 ml Erlenmeyer flask. Cells immobilized on polyurethane foam produced 25 nkat/ml/day of enzyme.

IRVINE et al. (U.S. Pat. No. 5,342,765) proposed a method in which by immobilizing Phanerochaete chrysosporium on an oxygen-permeable surface and by supplying oxygen to the organism through the oxygen-permeable surface, the production of the extracellular enzyme by the fungus developed to a level of 230 units/L (U/L) and averaged 121 U/L over 8 batch production periods. Veratryl alcohol (1 mM) was used along with the production medium to enhance lignin peroxidase production.

A proposal has been made (U.S. Pat. No. 5,972,672) on Phanerochaete chrysosporium immobilized in bioreactor and supplied by very complicate culture medium including phospholipids and veratryl alcohol. In addition, a stream of pure oxygen bubbles was supplied at a flow rate of 20 L/h. Under these conditions maximum MnP and LiP activities were 10000 U/L and 2400 U/L, respectively.

Call (U.S. Pat. No. 5,403,723) used Cerrena unicolor as laccase producer. Very complex and expensive medium was developed for fungus cultivation. Besides the main medium components it contained cells of yeasts or benzaldehydes, chlorobenzaldehydes, nitrobenzaldehydes, hydroxybenzaldehydes, aminobenzaldehydes, methylbenzaldehydes, diaryls or triaryls, dialkylalkanes, trialkyl alkanes, open-chained or cyclic imines or derivatives of the aforementioned substances as inductive compounds. Each day, the flasks were gassed with O.sub.2 for 30 seconds (100 l/hour). The cultivation period was 4-5 days. The enzyme yields are approx. 1500-2000 IU/l.

However, the yields or levels of the ligninolytic enzymes production in these and other proposals are still unsatisfactory to make a large-scale commercial production profitable. They use very expensive equipment and medium composition; some of inducers used are toxic. Accordingly, to develop commercially viable and low cost technologies of ligninolytic enzyme production there is a need for fungus with over-expression enzyme activity and for the procedure ensuring highest enzyme activity using simple, not toxic medium and rapid, inexpensive manufacturing processes. The process of present invention involves the production of ligninolytic enzymes from white rot fungi in submerged cultivation on nutrient media especially formulated to produce high yields of target enzymes using fermentation of lignocellulosic agro-industrial wastes.

### SUMMARY OF THE INVENTION

It was the objective of the inventor to increase the yield and decrease the cost of extracellular laccase and manganese peroxidases produced by white rot basidiomycetes. For this purpose, we succeeded in detecting new strains (Cerrena unicolor and Trametes versicolor) that are hypersecretory of the laccase and manganese peroxidase. We have discovered a superior medium for producing extracellular enzymes from basidiomycetous fungi, particularly for producing laccase and manganese peroxidase from the aforesaid white rot fungi. To this end, submerged fermentation techniques using lignocellulosic substrates like waste of ethanol production from wheat grain, mandarin peels, bran and others, have been developed. In particular, we devised specific culture conditions, which can be carried out in fermentor so that the enzyme yield is drastically increased. We have found that it is preferable to create at first appropriate culture conditions (agitation, aeration, pH of medium) that maintain best growth of fungi during the first 2-3 days, while the culture conditions set at the second stage of fermentation serve for a predominant synthesis of the target enzymes. This enables to considerably increase the yield of laccase and manganese peroxidase as compared to that obtained with the cultures of the prior art. Implementation of the present invention makes it possible, in addition, to control and modify the laccase/manganese peroxidase ratio in accordance with requirements.

### DETAILED DESCRIPTION OF THE INVENTION

The overall object of the present invention is twofold. The first aspect of the invention is white rot fungus particularly Trametes versicolor and Cerrena unicolor, that were isolated in Georgia (former USSR Republic) and deposited in the Culture Collection of the Netherlands (Centraalbureau voor Schimmelcultures, International Depository Authority, and temporary references: Trametes versicolor CBS 117346 and Cerrena unicolor CBS 117347).

The second aspect of the present invention is a process for producing laccase and/or manganese peroxidase from cultures of the aforesaid white rot fungi, which process is characterized by comprising the culturing of at least one strain chosen from the group consisting of the strains mentioned above.

The basics of the above cultures' production rely on the standard techniques of basidiomycetes culture growing practice, which are known per se, from an inoculum consisting of homogenized mycelia fragments obtained from a pre-culture. The method is applicable to any aerobic microorganisms, including bacteria, fungi, and yeasts, which possess lingocellulolytic enzyme system and thus have the capability to degrade lignocellulose for nutrition.

Examples of fungi that can be used in the process of this invention include the white rot fungi, such as Trametes versicolor, Phanerochaete chrysosporium, Phlebia radiata, Funalia trogii, Cerrena unicolor, which produce laccase and/or manganese peroxidase and/or lignin peroxidase. The process of this invention can be used to produce a variety of extracellular products including, for example, various enzymes, antibiotics, alcohols, pharmaceuticals, hormones and proteins.

In order to produce ligninolytic enzymes, the culture of white rot fungi is treated in aqueous media such as those employed for good mycelium growth and biomass accumulation. The culture medium comprises at least one lignocellulosic substrate and at least one nitrogen source, inorganic salts and it is supplemented with yeast extract.

All species of white rot fungi are capable to utilize lignocellulose materials, so a wide range of carbohydrates including pentoses, hexoses and polysaccharides are good sources of carbon and energy for their growth. The concentration of the lignocellulosic substrate is preferably between 30 and 80 g/L. A lignocellulosic substrate preferably used comprises ethanol production wastes, mandarin peels and bran, but other lignocellulosic substrates can be also used, stand-alone or mixed.

The nitrogen source can, for example, consist of ammonium nitrate, ammonium tartrate or peptone in combination with yeast extract. The concentration of the nitrogen source is preferably between 0.5 and 5 g/L.

Among inorganic salts, which can be incorporated in the culture medium are different salts that contain cations of potassium and magnesium. Useful cations can be obtained in the form of phosphate, sulphate and chloride. The assimilable inorganic salts are used at a concentration of between 0.2 and 1 g/L.

No special activating and/or protective components, trace elements or vitamins are used in our processes.

To carry out the process according to the invention, culturing may be performed in a manner known per se, either in flasks or in fermentor. The submerged fermentation includes one or more stages of seed development under controlled conditions. The liquid nutrient medium for the first step of inoculum preparation may be any suitable combination of carbon and nitrogen source, preferably glucose, peptone and yeast extract. The flasks containing 100 ml of nutrient medium are inoculated from surface agar culture (tube or Petri dish) and they are cultivated on shaker at 140 rpm and 27° C. After 5-6 days, the fungus mycelium is homogenized in a Waring laboratory blender and mycelium homogenate is transferred in to sterile medium in the proportion 1:5-1:20. In this case, the mycelium is grown in the form of balls (pellets), which can be from 0.5 to 5 mm in diameter.

The fermentor employed is a bench stirred tank reactor (diameter/height ratio d/h=1/2 to 1/3 approx.) that possesses a stirrer module, for example, a Rushton blade impeller.

In one advantageous mode of carrying out the method of producing ligninolytic enzyme according to the present invention, the first step of fermentation takes place for a period of about 2-3 days in a culture medium without pH control (to provide good growth of fungus), whereas the next step is carried out in a culture medium controlled at pH 5.5.

The culturing is preferably performed with aeration and agitation of the medium.

Aeration of the medium is carried out by introducing air, pure oxygen or any other mixture of gases for ensuring a sufficient supply of oxygen to the fungus, by means of a device that allows a homogeneous dispersion of this gas.

Agitation of the medium may be performed mechanically. It can also be done pneumatically by using a direct action of the aeration system, or by an equivalent system used simultaneously.

The level of agitation and/or of aeration is found a way to permit the formation of mycelial pellets of an average diameter 0.5 to 5 mm, while limiting the shearing stresses undergone by the fungus biomass. This level may be variable during the culture period.

The cultivation should preferably be carried out at the temperature of approximately 27° C.

The production of laccase/manganese peroxidase mixture as well as the Lac/MnP ratio can be controlled in accordance with the substrate and the strain used. It is thus possible to obtain enzyme cocktails with a desired predominance of either laccase or manganese peroxidase.

To modify the Lac/MnP ratio in favour of the production of laccase, the culturing of Cerrena unicolor, for instance, is carried out in the presence of an ethanol production waste at a concentration of between 30 and 60 g/L, preferably of the order of 50 g/L.

To modify the Lac/MnP ratio in favour of the production of manganese peroxidase, the culturing of Cerrena unicolor, for instance, is carried out in the presence of the mandarin peels at a concentration of between 30 and 80 g/L, preferably of the order of 60 g/L.

The method according to the present invention leads to a considerable increase in the laccase and manganese peroxidase activity and productivity as compared to all the methods known hitherto. In fact, whereas a laccase activity of the order of 500-600 U.l.sup.-1 is obtained after 14 days fungi cultivation in the case in which a complex culture medium of Cerrena unicolor is supplemented with inductors (veratric acid, ferulic acid or xylidine), and whereas the manganese peroxidase activity is of the order of 30-300 U.l.sup.1 when the culture medium is supplemented with inductors (ROGALSKI J., DAWIDOWICZ A., JOZWIK E., LEONOWICZ A. IMMOBILIZATION OF LACCASE FROM CERRENA UNICOLOR ON CONTROLLED POROSITY GLASS. J. MOL. CATALYSIS B: ENZYMATIC. 1999, 6, 29-39; GIL P. K., ARORA D. S. EFFECT OF CULTURE CONDITIONS ON MANGANESE PEROXIDASE PRODUCTION AND ACTIVITY BY SOME WHITE ROT FUNGI. J. IND. MICROBIOL. BIOTECHNOL. 2003, 30, 28-33), the laccase and manganese peroxidase activities reach 400000 U.l.sup.-1 and 7000 U.l.sup.-1, respectively, after 5-6 days of, say, Cerrena unicolor cultivation when using the method of the present invention, in which culture medium is supplemented with selected lignocellulosic substrate, mandarin peels or ethanol production waste.

Depending on the applications, the culture liquid generating enzyme cocktails of laccase and/or manganese peroxidase may be used directly, or after concentration, performed, for example, by ultrafiltration. For the purposes of purification, ion exchange and size exclusion chromatography is carried out. The laccase variant secreted from the host cells may conveniently be recovered from the culture medium by well-known procedures or their variants, including separating the cells from the medium by centrifugation or filtration, and precipitating protein components of the medium by means of a salt such as ammonium sulphate, followed by use of chromatographic procedures such as ion exchange chromatography, affinity chromatography, or the like.

The following examples further illustrate this invention. It should be clearly understood, however, that these examples are given only by way of illustration of the subject of the invention, of which they in no way constitute a limitation.

### EXAMPLE 1

Cerrena unicolor is used as producing strain. First, malt extract agar is inoculated and then cultivated at 27° C. for approximately 10-12 days. Amount of 1-2 cm² of grown mycelium is removed from the surface of agar and inoculated in a 100 ml of nutrition medium in a 500 ml Erlenmeyer flask (cultivation time approximately 5 days at 27° C.). The pre-culture thus obtained is then homogenized in a Waring laboratory blender for 30 seconds twice. 50 ml of the pre-culture mycelium homogenate are added per litre of medium in the shaking flasks, i.e. 25 ml are added to a 2 l shaking flasks being filled with 0.5 L of medium containing ethanol production waste. The cultivation temperature is 27° C.; the shaking frequency is 140 rpm. The cultivation period is 7-10 days. The laccase and manganese peroxidase yields are approximately 50000-150000 IU/L and 500-800 IU/L, respectively.

The medium composition is as follows:

a) Pre-culture medium (g/L):

Glucose—10.0

Peptone—0.2

Yeast extract—0.3

KH.sub.2 PO.sub.4—0.8

Na.sub.2 HPO.sub.4—0.2

MgSO.sub.4.times.7 H.sub.2 O—0.5

b) The main medium (g/L):

Waste from ethanol production from wheat grain—50.0

Peptone—0.3

Yeast extract—0.5

KH.sub.2 PO.sub.4—0.8

Na.sub.2 HPO.sub.4—0.2

MgSO.sub.4—0.5

### EXAMPLE 2

### The Conditions of Example 1 are Repeated; 0.5 L of Main Nutrition Medium Contains 60 g/L of Milled Mandarin Peels).

Cerrena unicolor is used as a strain. First, malt extract agar is inoculated and then cultivated at 27° C. for about 10-12 days. 1-2 cm² of grown mycelium is removed from the surface of agar and is inoculated in a 100 ml of nutrition medium in a 500 ml Erlenmeyer flask (cultivation time approximately 5 days at 27° C.). The pre-culture thus obtained is then homogenized in a Waring laboratory blender twice for 30 seconds. 50 ml of pre-culture mycelium homogenate are added per litre of medium in the shaking flasks, i.e. 25 ml are added to a 2 L shaking flasks being filled with 0.5 L of medium containing milled mandarin peels. The cultivation temperature is 27° C.; the shaking frequency is 140 rpm. The cultivation period is 6-9 days. The laccase and manganese peroxidase yields are approximately 15000-25000 IU/L and 4000-7000 IU/L, respectively.

The medium is composed as follows:

a) Pre-culture medium (g/l):

Glucose—10.0

Peptone—0.2

Yeast extract—0.3

KH.sub.2 PO.sub.4—0.8

Na.sub.2 HPO.sub.4—0.2

MgSO.sub.4 .times.7 H.sub.2 O—0.5

b) The main medium (g/l):

Milled mandarin peels—60.0

Peptone—0.3

Yeast extract—0.5

KH.sub.2 PO.sub.4—0.8

Na.sub.2 HPO.sub.4—0.2

MgSO.sub.4—0.5

### EXAMPLE 3

Trametes versicolor is used as a strain. First, malt extract agar is inoculated and then cultivated at 27° C. for approximately 10-12 days. 1-2 cm² of grown mycelium is removed from the surface of agar and is inoculated in a 100 ml of nutrition medium in a 500 ml Erlenmeyer flask (cultivation time approximately 5 days at 27° C.). The pre-culture thus obtained is then homogenized in a Waring laboratory blender for 30 seconds twice. 50 ml of pre-culture mycelium homogenate are added per litre of medium in the shaking flasks, i.e. 25 ml are added to a 2 L shaking flasks being filled with 0.5 L of medium containing milled mandarin peels. The cultivation temperature is 27° C.; the shaking frequency is 140 rpm. The cultivation period is 4-5 days. The laccase and manganese peroxidase yields are approximately 15000-20000 IU/L and 200-400 IU/L, respectively.

The medium is composed as follows:

a) Pre-culture medium (g/l):

Glucose—10.0

Peptone—0.2

Yeast extract—0.3

KH.sub.2 PO.sub.4—0.8

Na.sub.2 HPO.sub.4—0.2

MgSO.sub.4 .times.7 H.sub.2 O—0.5

b) The main medium (g/l):

Milled mandarin peels—60.0

Peptone—0.3

Yeast extract—0.5

KH.sub.2 PO.sub.4—0.8

Na.sub.2 HPO.sub.4—0.2

MgSO.sub.4—0.5

### EXAMPLE 4

Cerrena unicolor is used as a strain. First, malt extract agar is inoculated and then cultivated at 27° C. for approximately 10-12 days. 1-2 cm² of grown mycelium is removed from the surface of agar and is inoculated in a 100 ml of nutrition medium in a 500 ml Erlenmeyer flask (cultivation time approximately 5 days at 27° C.). The pre-culture thus obtained is then homogenized in a Waring laboratory blender for 30 seconds twice. 50 ml of pre-culture mycelium homogenate are added per litre of medium in the shaking flasks, i.e. 25 ml are added to a 2 L shaking flasks being filled with 0.5 l of pre-culture medium. The cultivation temperature is 27° C. the shaking frequency is 140 rpm. The cultivation period is 5 days, and then biomass is homogenized in a Waring laboratory blender for 30 seconds twice. 50 ml of the second pre-culture mycelium homogenate are added per litre of medium in the shaking flasks, i.e. 400 ml are added to a 12 L bench stirred tank Bioflo 2000 fermentor (New Brunswick Scientific Co., N.J.) being filled with 8 L of medium containing lignocellulosic waste after ethanol production from wheat grains.

Initial parameters of cultivation are as follows: agitation—150-300 rpm; DO—10%; pH—free variable regime; aeration—0.25 v/v/min, temperature—27° C. The antifoam used is polypropylene glycol 2000. 4% KOH and 4% HCl that are used to control pH during fermentation. After 48-72 h, the airflow rate is increased to 0.5 L/min and medium pH is controlled at 5.5. After 96 hours, the agitation is decreased to 200 rpm and cultivation is continued up to 6 days. The laccase and manganese peroxidase yields are approximately 200000-400000 IU/L and 200-600 IU/L, respectively.

The medium composition:

a) Pre-culture medium (g/l):

Glucose—10.0

Peptone—0.2

Yeast extract—0.3

KH.sub.2 PO.sub.4—0.8

Na.sub.2 HPO.sub.4—0.2

MgSO.sub.4 .times.7 H.sub.2 O—0.5

b) The main medium (g/l):

Lignocellulosic waste after ethanol production from wheat grains—50.0

Peptone—0.3

Yeast extract—0.5

KH.sub.2 PO.sub.4—0.8

Na.sub.2 HPO.sub.4—0.2

MgSO.sub.4—0.5

1-7. (canceled)

8. A wood-rotting Basidiomycetes fungus, Trametes vesicolor having an International Depository Authority Number CBS1 17346.

9. A process for manufacturing of ligninolytic enzymes, said process comprising:
(1) providing a submerged fermentation system filled with an aqueous medium containing growth-stimulating nutrients and the wood-rotting Basidiomycetes fungus of claim 8 that extracellulary produces laccase and peroxidases;
(2) maintaining two sets of fermentation parameters: first one for ensuring optimal growth of fungi for at least 2-3 days while the second set of parameters serves for optimising the best enzyme synthesis;
(3) isolating said ligninolytic enzymes from said aqueous medium.

10. A ligninolytic enzyme derived from the wood-rotting Basidiomycetes fungus of claim 8.

11. The ligninolytic enzyme of claim 10, being selected from the group consisting of a laccase and a manganese peroxidase.

12. A method for bioremediation comprising contacting waste with the wood-rotting Basidiomycetes fungus of claim 8, or an enzyme derived therefrom.

13. The method of claim 12, wherein said enzyme is selected from the group consisting of a laccase and a manganese peroxidase.

14. A method of biobleaching comprising contacting a substance with the wood-rotting Basidiomycetes fungus of claim 8, or an enzyme derived therefrom, thereby generating a biobleached substance.

15. The method of claim 14, wherein said enzyme is selected from the group consisting of a laccase and a manganese peroxidase.

16. The method of claim 14, wherein said substance comprise textile.

17. A method of biopulping comprising contacting a lignocellulosic substrate with the wood-rotting Basidiomycetes fungus of claim 8, or an enzyme derived therefrom, thereby biopulping the lignocellulosic substrate.

18. The method of claim 17, wherein said enzyme is selected from the group consisting of a laccase and a manganese peroxidase.

19. A wood-rotting Basidiomycetes fungus, Cerrena unicolor having an International Depository Authority Number CBS117347.

20. A process for manufacturing of ligninolytic enzymes, said process comprising:
(1) providing a submerged fermentation system filled with an aqueous medium containing growth-stimulating nutrients and the wood-rotting Basidiomycetes fungus of claim 19 that extracellulary produces laccase and peroxidases;
(2) maintaining two sets of fermentation parameters: first one for ensuring optimal growth of fungi for at least 2-3 days while the second set of parameters serves for optimising the best enzyme synthesis;
(3) isolating said ligninolytic enzymes from said aqueous medium.

21. A ligninolytic enzyme derived from the wood-rotting Basidiomycetes fungus of claim 19.

22. The ligninolytic enzyme of claim 21, being selected from the group consisting of a laccase and a manganese peroxidase.

23. A method for bioremediation comprising contacting waste with the wood-rotting Basidiomycetes fungus of claim 19, or an enzyme derived therefrom.

24. The method of claim 23, wherein said enzyme is selected from the group consisting of a laccase and a manganese peroxidase.

25. A method of biobleaching comprising contacting a substance with the wood-rotting Basidiomycetes fungus of claim 19, or an enzyme derived therefrom, thereby generating a biobleached substance.

26. The method of claim 25, wherein said enzyme is selected from the group consisting of a laccase and a manganese peroxidase.

27. The method of claim 25, wherein said substance comprise textile.

28. A method of biopulping comprising contacting a lignocellulosic substrate with the wood-rotting Basidiomycetes fungus of claim 19, or an enzyme derived therefrom, thereby biopulping the lignocellulosic substrate.

29. The method of claim 28, wherein said enzyme is selected from the group consisting of a laccase and a manganese peroxidase.
